(19) United States
(12) Patent Application Publication
(10) Pub. No.: US 2008/0275486 A1
(43) Pub. Date: Nov. 6, 2008

(54) EXTENDED DURATION MEDICAL FILTER

(76) Inventors: Clifford Dwyer, Weston, FL (US); George Landau, Huntington, PA (US)

Correspondence Address: RONALD L. HOFER, 122 LINDBERGH LANE, MOORESVILLE, NC, 28117, US

(21) Appl. No.: 11/742,681

(22) Filed: May 1, 2007

(51) Int. Cl. A61M 29/00

(52) U.S. Cl. 606/200

(57) ABSTRACT

A medical filter for placement inside a body passage such as a vessel has a first filter half and a second filter half each of which have generally longitudinally extending legs with free ends. The first filter half has a docking station centrally located at one longitudinal end and a stop carried by one of said legs centrally located at its other end while the second filter half has a cylindrical collar and a longitudinally centrally located hook. The filter halves fit together with legs in interlocking relationship and the docking station has a smaller diameter than the diameter of the collar so that the filter can be retrieved by pushing the first filter half into the collar of the second filter half with a mandrel pushing against the docking station while holding the second filter half with a snare attached to its hook to collapse the first filter half in the collar of the second filter half and then pulling the second filter half into the lumen of a retrieval catheter with the snare.

### BACKGROUND OF THE INVENTION

The present invention relates to medical filters which are intended to be placed inside a blood vessel or other body passage for the purpose of intercepting thrombus or other particulate matter.

Medical filters, including vena cava filters are generally known and are used to intercept unwanted particulate material in blood vessels and the like. It has been found that certain features are desirable in such filters. It is desirable that the filter be designed so that it can be emplaced with minimal trauma to the patient as by percutaneous delivery. It is also desirable that the filter be of a design which can adapt or adjust to the size of the vessel. To function satisfactorily in a blood vessel, a filter must be stable and effective to entrap thrombus, clots or other dangerous coagulations while allowing free flow of blood in the vessel. In some cases it is desirable to have a filter which can be inserted in a vessel for longer term emplacement, i.e. beyond two weeks, and then be removed with minimal trauma to the vessel wall.

While medical filters such as vena cava filters have been known and used for several years, there remains room for improvements in their designs and methods of emplacement and retrieval. Conventionally, such filters are delivered in a compressed form in the lumen of a delivery catheter and are retrieved by using a snare to withdraw the filter into the lumen of a retrieval catheter. However, difficulties continue to be encountered with presently known filters and there remains room for improved designs of filters and their methods of delivery and removal. In particular, there remains a need for filters with improved implantation and retrieval characteristics.

Accordingly, the present invention provides a medical filter which can be inserted in a body passage for a long period of time and then removed if desired with minimal trauma to the cells of the wall off the body passage. The filter of the present invention can be emplaced and retrieved from either femoral or jugular approaches. Furthermore, the design is practical for manufacture and can be economically made.

Further understanding of the present invention will be had from the following description taken in conjunction with the accompanying drawings and appended claims.

### BRIEF DESCRIPTION OF THE DRAWINGS

FIG. 1 is a side elevation of a preferred embodiment of a filter of the present invention shown in expanded form and in operative association with a body vessel shown in side sectional view;

FIG. 2 is a side elevation showing the two halves of the filter of FIG. 1 separated from each other and in expanded form;

FIG. 3 is a plan view of the two halves of the filter of FIG. 1 separated from each other and in expanded form;

FIG. 4 is a side elevation with portions broken away, of the preferred embodiment of FIG. 1 shown in compressed form in a delivery catheter; and

FIG. 5-8 are somewhat schematic views illustrating steps of a preferred method of retrieval of the filter of FIG. 1 from a body vessel;

### SUMMARY OF THE INVENTION

In accordance with the present invention, a medical filter for placement inside a body passage such as a vessel comprises:
(A) a first filter half defining a first filter basket having an open end and having a radially centrally located docking station with legs extending generally radially and axially therefrom towards said open end of said first filter basket;
(B) a second filter half defining a second filter basket having an open end facing in a direction opposite to that of the open end of said first filter basket and having a radially centrally located collar with legs extending generally radially and axially therefrom towards said open end of said second filter basket, said second filter basket having a retrieval element;
   an end portion of said first filter half being sized to fit within said collar of said second filter half in coaxial relationship.

In accordance with a preferred method of retrieval of the present invention, the medical filter of the present invention is retrieved by steps of:
(A) attaching a snare to the retrieval element of the second filter half,
(B) exerting a pushing force with a mandrel against the docking station of the first filter half while holding the second filter half with the snare attached in opposition to the pushing force in a direction to move the filter basket of the first filter half into and through the collar to thereby collapse the first filter basket therein; and
(C) pulling the second filter half with said first filter half in radially collapsed condition into a lumen of a retrieval catheter, thereby radially collapsing the arms of said second filter half therein.

### DETAILED DESCRIPTION OF A PREFERRED EMBODIMENT OF THE INVENTION

The following description of the preferred embodiment of the present invention is intended to be merely illustrative in nature, and as such, is not intended to limit in any way the present invention, its application, or uses. Numerous modifications may be made by those skilled in the art without departing from the true spirit and scope of the invention, for example, the preferred embodiment of the present invention is shown in use in a vein but may also be used in other vessels of body passages.

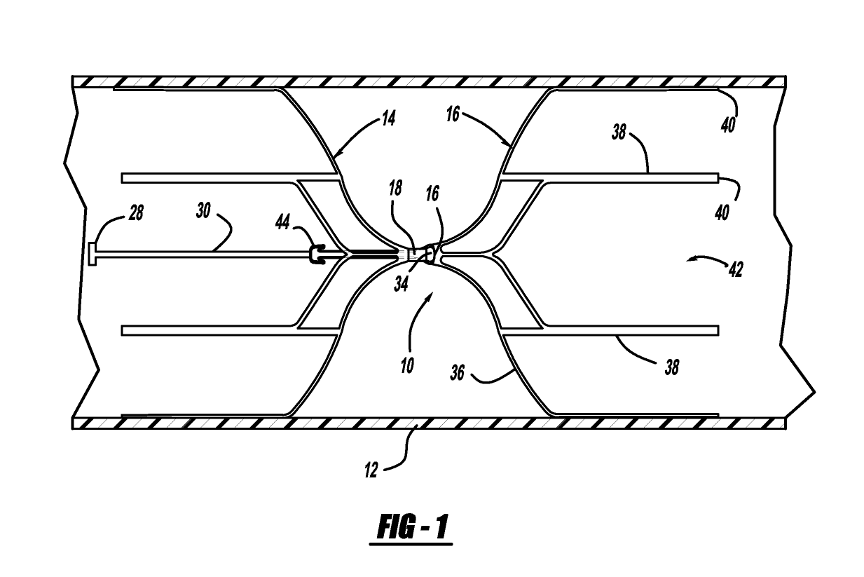

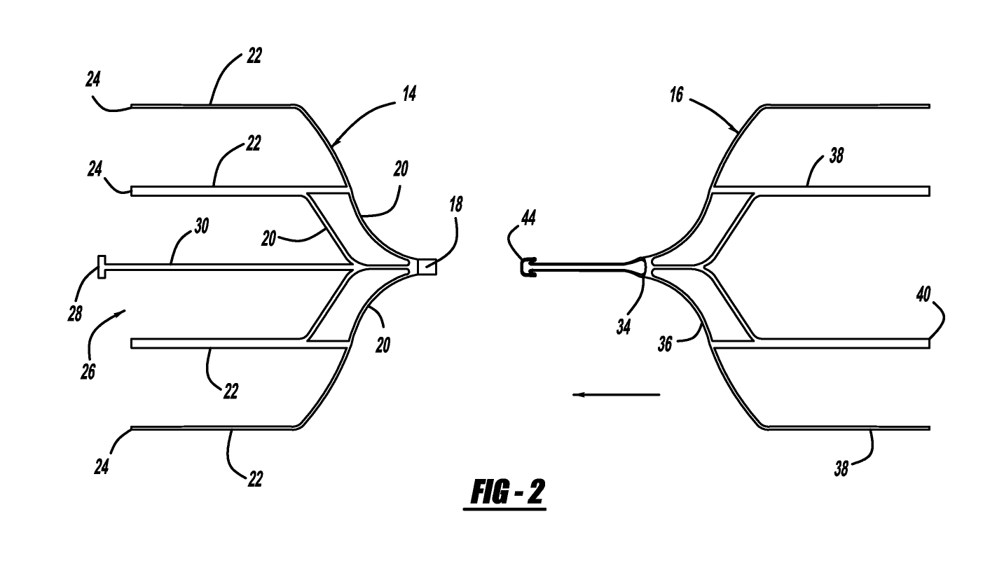

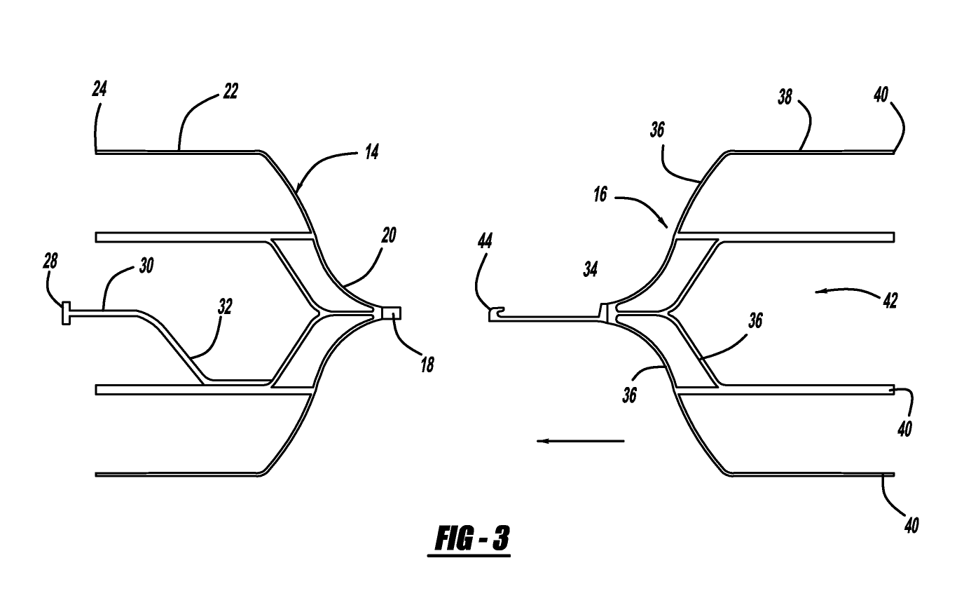

Now referring to FIG. 1, a preferred embodiment of a medical filter of the present invention is indicated generally by the numeral 10 and is shown in association with a blood vessel 12. Filter 10 is made of a resilient material which tends to expand to the form illustrated in FIG. 1 but can be compressed to a smaller diameter form as is conventional in the art. As best shown in FIGS. 2 and 3, filter 10 comprises two filter halves, first filter half 14 and second filter half 16, which, before assembly, are two separate pieces as shown in FIGS. 2 and 3.

First filter half 14 has a radially centrally located docking station 18 with a plurality of struts 20 extending generally radially outwardly and axially from docking station 18. Struts 20 connect or are integrally formed from one piece with generally parallel longitudinally extending legs 22. Legs 22 have free ends 24 and struts 20. Legs 22 define a first filter basket 26 with its open end open to the left as viewed in FIGS. 1-3. Each strut 20 can be secured to docking station 18 by any suitable attachment means or, alternatively, each strut 20 can be formed from a single piece of tubing along with docking station 18 to be integrally formed therewith. Struts 20 provide stability to legs 22 and, hence, fist filter half 14. Filter half 14 also has a stop 28 carried by arm 30 extending radially inwardly at 32 to position stop 28 on the longitudinal axis of filter half 14.

Second filter half 16 has a collar 34 with a plurality of struts 36 extending generally radially outwardly and axially from collar 34. Struts 36 connect or are integrally formed from one piece with generally parallel longitudinally extending legs 38. Legs 38 have free ends 40 and struts 36 and legs 38 define a second filter basket 42 with its open end open to the right as viewed in FIGS. 1-3. Collar 34 has a diameter large enough to receive docking station 18 and first filter basket 26 as is set forth in more detail below. Also, second filter half 16 has retrieval hook 44 positioned generally on its central axis and spaced longitudinally from collar 34 a distance to come into contacting relationship with stop 28 when first filter basket 42 is collapsed into collar 34 during retrieval of filter 10 as is described in more detail below. Of course, other designs of retrieval hook 44 are suitable for use herein and will be apparent to those skilled in the art. For example, retrieval hook 44 can be a single hook or a double hook or can be any retrieval element so long as it can function to allow retrieval of filter 10 in accordance with the present invention.

Filter baskets 26 and 42 act in opposition to flow of blood through blood vessel 12 and function to trap thrombus in the blood flowing therethrough. Filter halves 14 and 16 of Filter 10 are shown to be generally symmetric in form although it will be appreciated by those skilled in the art that asymmetric forms of filter halves 14 and 16 are within the broad scope of the present invention so long as their form does not interfere with their function as described hereinafter. For example, filter half 14 may have more or fewer legs 22 and/or struts 20 than filter half 16. Of course, filter halves 14 and 16 are not analogous with respect to docking station 18 and stop 28 of first filter half 14 or collar 34 and retrieval hook 44.

Each leg 22 and 38 extends, when not compressed, generally parallel to the longitudinal axis of filter 10, i.e., axially, and is not connected to any strut near blood vessel 12. Thus, each free end 24 and 40 of each leg 22 and 38 may become encapsulated by endothelium cell growth but no strut poses a substantial mechanical interference with the cell growth so as to make it difficult to retrieve filter 10 when retrieved in accordance with the method of this invention. Furthermore, legs 22 and 38 extend in opposite directions to thereby provide stability to filter 10 and resistance to migration of filter 10 in blood vessel 12 in either axial direction Of course, each filter half 14 and 16 is individually freely movable in opposite axial directions for retrieval as provided in the method of the present invention. Thus, in accordance with the present invention, the long narrow legs 22 and 38 stabilize filter 10 without becoming permanently embedded in the vessel wall by endothelium cell growth. While legs 22 and 38 may become covered with cell growth, the shape of the legs and their method of recapture allows the legs to slide out of the cell lining much like a needle would move through a vessel wall. There are no strut connections that connect the ends of legs 22 or 38 to each other at their free ends 24 and 40. Thus each leg 22 and 38 can easily slide out of the cell lining.

Filter 10 may be made of any suitable material and by a variety of methods. For example, Nitinol, stainless steel, elgiloy, cobalt chromium, or suitable polymeric material are suitable materials. Suitable methods of manufacture include cutting a pattern into a tube to enable expansion of the tube into the desired body and struts. Another suitable method is to form the struts and body from separate strips or wires and then to join the respective parts together by suitable methods which are well known in the art. Other materials may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter. Thus, the material must also be sufficiently resilient to accomplish both compression in the delivery catheter and expansion upon ejection from the catheter.

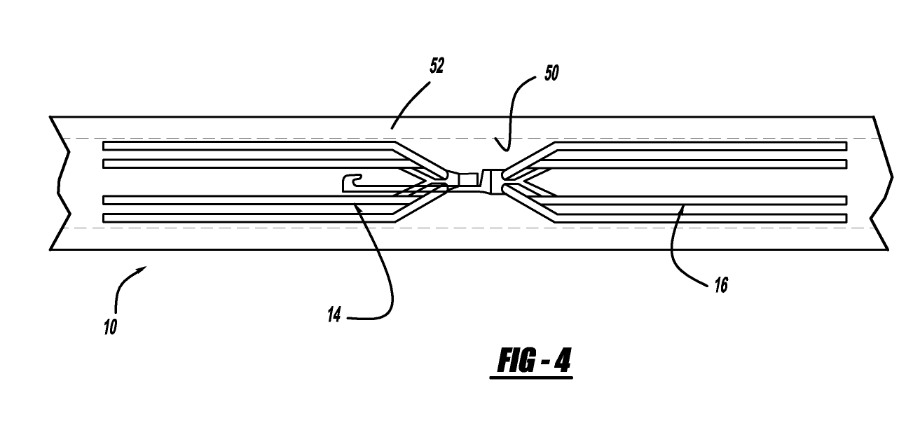

Filter 10 is intended to be initially deployed compressed in a delivery catheter. Referring to FIG. 4, filter 10 is shown in radially compressed form in lumen 50 of delivery catheter 52. Insertion of the filter into a delivery catheter can be by any conventional method and may be carried out by several different methods, including simply assembling filter halves 14 and 16 and then pushing filter 10 into the lumen at the distal end of a delivery catheter. Alternatively, filter 10 may be inserted into the proximal end of a delivery catheter and pushed by means of a push wire or the like to the distal end of the catheter. The delivery catheter may be inserted along a blood vessel using either a femoral approach or a jugular approach. A push wire can be used to eject the filter from the lumen of the catheter. Upon ejection from the delivery catheter, filter 10 self-expands into its operative form shown in FIG. 1.

Having described the structure of filter 10, a preferred embodiment of the present invention, further understanding of the unique character and advantages of the present invention will be had by an understanding its method of retrieval.

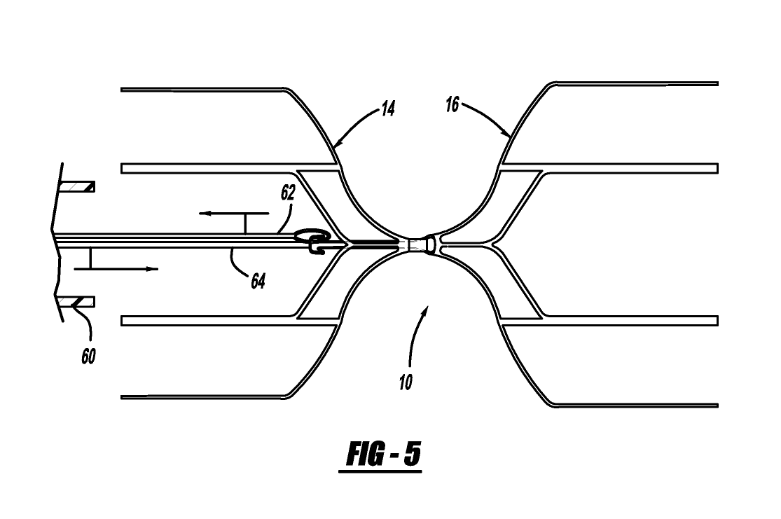

Now referring to FIGS. 5-8, retrieval of filter 10 from a vessel is illustrated somewhat schematically. FIG. 5 illustrates a first stage in a preferred method of retrieval of filter 10. As shown in FIG. 5, a retrieval catheter 60 has been inserted along a blood vessel (not shown) to a position proximate to the location of filter 10. Snare 62 from catheter 60 is used to engage in tension hook 44 of filter half 16. Catheter 60 also carries a stiff wire or mandrel 64 which is inserted through the lumen of catheter 60 into engagement in compression with the docking station 18 of first filter half 14. While holding second filter half 16 with snare 62 to prevent rightward movement as viewed in FIG. 5, mandrel 64 is advanced rightwardly as viewed in FIG. 5.

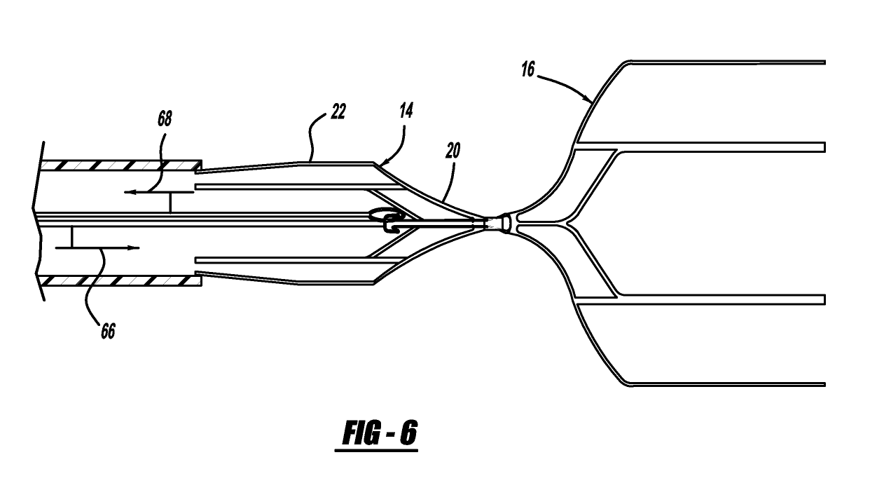

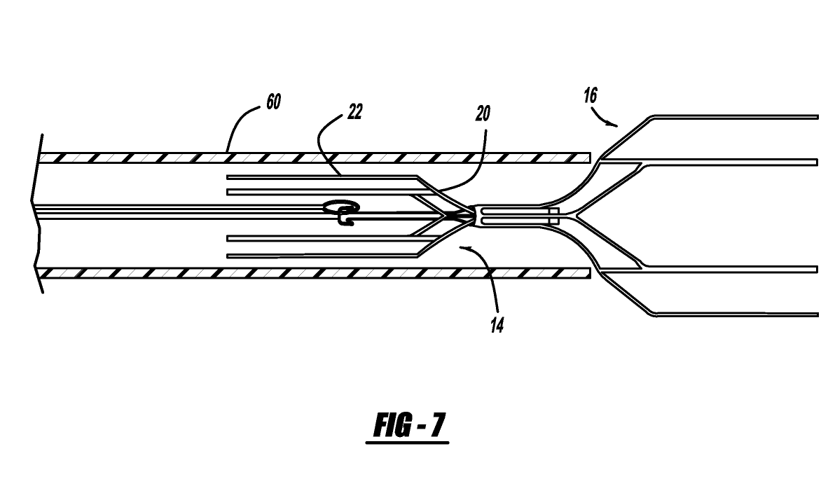

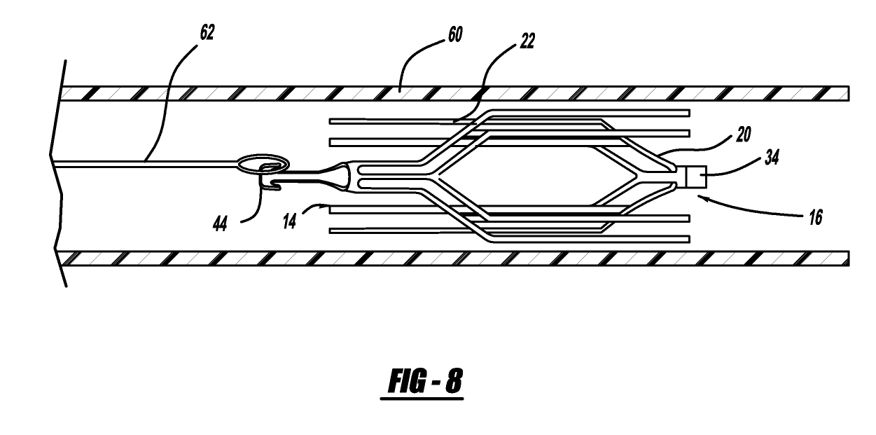

As illustrated in FIG. 6, rightward movement of mandrel 64 urges and moves filter half 14 to the right as indicated by arrow 66 with respect to filter half 16 which is held in tension in the direction indicated by arrow 68 by snare 62. The result of this relative movement between the filter halves is to draw down or compress struts 20 and legs 22 of first filter half 14 radially inwardly as they are pushed through the inner diameter of collar 44 of second filter half 16. As illustrated in FIG. 7, upon complete compression of first filter half 14, hook 44 comes into contact with stop 28. Then, and as illustrated in FIG. 8, second filter half 16, with first filter half 14 radially compressed and held coaxially within second filter half 16, can be withdrawn into retrieval catheter by pulling snare 62 leftward as viewed in FIG. 7 to pull arms 38 to the left and then radially inwardly as they are compressed into the inner lumen of retrieval catheter 60. Finally, filter 10 is inside catheter 60 as illustrated in FIG. 8 and can be removed from the vessel.

Thus, during retrieval of filter 10, first the proximal half of the filter is forced into the inner diameter of the distal half of the filter. As the proximal half end is fully recaptured it folds into the distal implant inner diameter and a stop is reached. The stop leg does not touch the lumen wall, it is shape set to extend to the middle of the implant or lumen. The legs of the proximal half slide out of the vessel lining, as the proximal half is recaptured. Once the proximal half is recaptured the snare pulls the distal (with the recaptured proximal half) inside the catheter.

A filter made in accordance with the present invention is retrievable at any time following implantation and may be implanted and retrieved from either femoral or jugular approaches.

The foregoing description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. It will be appreciated by those skilled in the art that the present invention is subject to variations and modifications. For example, the filter may be cut from a single tube and have end rings which are integral, i.e., one piece, with the spine and struts. The end rings may be tubular or cut into a serpentine pattern to allow the end rings to expandable to a larger diameter and subsequently compressible to a smaller diameter. This feature allows for very low profile filters when in the compressed state—an obvious advantage for insertion and removal. Of course, the filter may be tube-based or wire based or a combination.

1. A medical filter for placement inside a body passage to treat a patient, comprising:
(A) a first filter half having longitudinally extending legs with free ends, said first filter half having a docking station centrally located at one longitudinal end thereof;
(B) a second filter half having longitudinally extending legs with fee ends, said second filter half having a cylindrical collar and a longitudinally centrally located retrieval element;
said first and second filter halves being adapted to fit together in interlocking leg relationship and said docking station fitting within the diameter of said collar so that said first filter half can be collapsed through said collar.

2. A medical filter as in claim 1, wherein each said first and second filter half is a one-piece construction.

3. A medical filter as in claim 1, wherein said first filter half has a tubular section adjacent said docking station and said tubular section has a diameter less than the diameter of said collar.

4. A medical filter as in claim 1, wherein said hook is spaced from said collar and is located at the longitudinal end opposite the free ends of said legs of said second filter.

5. A medical filter as in claim 1, wherein said legs are comprised of wires.

6. A medical filter as in claim 1, wherein said legs are comprised of pieces cut from tubing.

7. A medical filter as in claim 1, wherein said first filter half has a stop element.

8. A method of retrieving a medical filter from a body passage of a patient such as from a vessel of a patient, said filter having a first filter half having longitudinally extending legs with free ends, said first filter half having a docking station centrally located at one longitudinal end thereof, and a second filter half having longitudinally extending legs with fee ends, said second filter half having a cylindrical collar and a longitudinally centrally located hook; said first and second filter halves being adapted to fit together in interlocking leg relationship and said docking station fitting within the diameter of said collar so that said first filter half can be collapsed through said collar, said method comprising the steps of:
(A) exerting a pushing force against said docking station and said first filter half in a direction to collapse said filter half through said collar while holding said second filter half with a snare attached to said hook in opposition to said pushing force; and
(B) pulling said second filter half with said first filter half in radially collapsed condition into a lumen of a retrieval catheter, thereby radially collapsing the arms of said second filter half therein.

9. The method of claim 8, wherein each said first and second filter half is a one-piece construction.

10. The method of claim 8, wherein said first filter half has a tubular section adjacent said docking station and said tubular section has a diameter less than the diameter of said collar.

11. The method of claim 8, wherein said hook is spaced from said collar and is located at the longitudinal end opposite the free ends of said legs of said second filter.

12. The method of claim 8, wherein said legs are comprised of wires.

13. The method of claim 8, wherein said legs are comprised of pieces cut from tubing.
